(19) United States
(12) Patent Application Publication
(10) Pub. No.: US 2025/0082585 A1
(43) Pub. Date: Mar. 13, 2025

(54) METHOD FOR STORING REDUCED COENZYME Q10

(71) Applicant: KANEKA CORPORATION, Osaka-shi, Osaka (JP)

(72) Inventors: Yuichi YOKOCHI, Takasago-shi (JP); Shiro KITAMURA, Osaka-shi (JP); Yuka FUKUYAMA, Takasago-shi (JP)

(73) Assignee: KANEKA CORPORATION, Osaka-shi, Osaka (JP)

(21) Appl. No.: 18/723,030

(22) PCT Filed: Dec. 21, 2022
(86) PCT No.: PCT/JP2022/047012

§ 371 Date: Jun. 21, 2024

(30) Foreign Application Priority Data
Dec. 24, 2021 (JP) 2021-210678
Sep. 26, 2022 (JP) 2022-152299

(51) Int. Cl. A61K 31/122; A61K 9/10

(52) U.S. Cl. CPC A61K 31/122; A61K 9/10

(57) ABSTRACT

The present description discloses a preferred solution for controlling oxidation of reduced coenzyme Q10 (QH) without any need for formulation of QH. One or more embodiments of the present invention relate to a method for storing QH, including storing a composition containing solid QH and water, a method for controlling oxidation of QH, including storing a composition containing solid QH and water, and a liquid composition containing solid QH in water. The solid QH is preferably one or more selected from the group consisting of a Form I crystal, a Form II crystal, an amorphous solid, and a co-crystal composed of QH and one or more other compounds.

### TECHNICAL FIELD

One or more embodiments of the present invention relate to a method for storing reduced coenzyme Q10, a method for controlling oxidation of reduced coenzyme Q10, and a liquid composition comprising reduced coenzyme Q10.

### BACKGROUND ART

Coenzyme Q is an essential component widely distributed in living organisms from bacteria to mammals, and is known as a member of mitochondrial electron transfer system in cells in the living organisms. The major component in humans is coenzyme Q10 having 10 repeating structures in the side chain of coenzyme Q, and usually, about 40% to 90% thereof is present in the living body as the reduced form. The physiological activity of coenzyme Q includes activation of energy production by mitochondrial activation, activation of cardiac function, stabilization of cell membranes, and protection of cells by antioxidant activity.

While coenzyme Q10 currently produced and sold is, in large part, oxidized coenzyme Q10, reduced coenzyme Q10 (hereinafter sometimes referred to as “QH”) which exhibits higher oral absorbability than that of oxidized coenzyme Q10 has also been commercially available and has come to be used in recent years.

Reduced coenzyme Q10 is easily oxidized, which leads to a problem that the storage cost is high and the scope of application of commercial products is restricted.

A common method for obtaining reduced coenzyme Q10 has already been disclosed (Patent Literature 1). Patent Literature 2 reports that crystal polymorphism is found in reduced coenzyme Q10. Patent Literature 2 reports that a newly appearing crystal form (wherein this crystal is hereinafter referred to as a “reduced coenzyme Q10 Form II crystal” or “QH Form II crystal”) is much more stable than the conventional reduced coenzyme Q10 (wherein this crystal is hereinafter referred to as a “reduced coenzyme Q10 Form I crystal” or “QH Form I crystal”) and also has other excellent physical properties.

Examples of the literature disclosing a technique for controlling oxidation of reduced coenzyme Q10 and stably storing reduced coenzyme Q10 include Patent Literature 3, Patent Literature 4, and Patent Literature 5. These Patent Literature describe, as reduced coenzyme Q10 having high oxidation stability and high in vivo absorbability, a particulate composition in which an oily component containing reduced coenzyme Q10 or an oily component containing reduced coenzyme Q10 and a lipophilic antioxidant forms a domain and is poly-dispersed in a matrix containing a water-soluble excipient or a matrix containing a water-soluble excipient and water-soluble ascorbic acids, and describe a method for stabilizing the particulate composition by placing the particulate composition in an ambient environment having a relative humidity of 90% or less. Examples of the water-soluble excipient described include gum arabic and gelatin. All of Patent Literature 3 to 5 disclose a technique for increasing oxidation stability by coating QH with a coating film of a gas barrier material such as gum arabic or gelatin.

Organic compounds are known to be generally less stable at a higher relative humidity (Non-Patent Literature 1 and 2).

Coenzyme Q10 is a hydrophobic compound. In order to formulate coenzyme Q10 in aqueous liquid compositions such as beverages, conventional techniques for solubilizing coenzyme Q10 in water have been developed. For example, Patent Literature 6 describes a water-soluble composition in which coenzyme Q10 is solubilized in water by emulsification. Patent Literature 7 describes a coenzyme Q10-containing liquid composition obtained by dispersing and emulsifying coenzyme Q10 in an aqueous liquid containing a water-soluble substance composed of starch octenylsuccinate and dextrin, and glycerin. Patent Literature 8 describes an emulsified composition in which coenzyme Q10 is emulsified, together with medium chain triglyceride, a surfactant and a polyhydric alcohol, in water. Patent Literature 9 describes a coenzyme Q-containing water-soluble composition containing coenzyme Q, hydrophilic polyglycerin fatty acid ester, lipophilic sucrose fatty acid ester, and an aqueous phase component.

Patent Literature 10 describes a co-crystal containing reduced coenzyme Q10 and a compound such as 3,4-dihydroxybenzoic acid, which has been found as an additional form of reduced coenzyme Q10. Patent Literature 11 describes formation of a co-crystal of reduced coenzyme Q10 and nicotinamide. Although reduced coenzyme Q10 may have increased oxidation stability due to co-crystallization of reduced coenzyme Q10 and one or more other compounds, even such co-crystallization cannot completely prevent reduced coenzyme Q10 from being oxidized.

### CITATION LIST

### Patent Literature

Patent Literature 1: JP Patent Publication No. H10-109933
Patent Literature 2: WO2012/176842
Patent Literature 3: WO2007/148798
Patent Literature 4: WO2008/129980
Patent Literature 5: JP Patent Publication No. 2009-149584 A
Patent Literature 6: JP Patent Publication No. 2004-196781 A
Patent Literature 7: WO2006/022187
Patent Literature 8: WO2006/035900
Patent Literature 9: WO2006/134970
Patent Literature 10: WO2019/162429
Patent Literature 11: Chinese Patent Publication No. 113024362 A

### Non-Patent Literature

Non-Patent Literature 1: Evaluating Stability of Vitamin C in Fortified Formula Using Water Activity and Glass Transition, Sablani S S, Al-Belushi K, Al-Marhubi I, and Al-Belushi R, International Journal of Food Properties, 2007, 10 (1): 61-71
Non-Patent Literature 2: Preformulation Studies of a Prodrug of Δ9-Tetrahydrocannabinol, Thumma S, Majumdar S, ElSohly M A, Gul W, and Repka M A, 2008, AAPS Pharm Sci Tech, 9 (3): 982-990

### SUMMARY OF INVENTION

### Technical Problem

Patent Literature 3 to 5 disclose a QH product that prevents oxidation of reduced coenzyme Q10 (QH) and can be stably stored. However, QH is required to be formulated with a specified component in all of Patent Literature 3 to 5, and thus applications of QH are limited.

Patent Literature 6 to 9 disclose a liquid composition in which coenzyme Q10 is solubilized in water by use of a surfactant or the like. However, such solubilized coenzyme Q10 cannot be separated again as a solid, and thus applications of coenzyme Q10 are limited. The purpose of the solubilization technique and the liquid composition described in Patent Literature 6 to 9 is to improve absorbability of (oxidized) coenzyme Q10, and oxidation stability of QH is not considered at all.

One or more embodiments of the present invention provide a method for storing QH, a method for controlling oxidation of QH, and a liquid composition containing QH, which are capable of controlling oxidation of QH without requiring formulation of QH.

### Solution to Problem

As described above, organic compounds are generally less stable at a higher humidity. Meanwhile, the present inventors have unexpectedly found that solid QH has high oxidation stability in the presence of water, and have completed the following respective aspects of the present invention.
(1) A method for storing reduced coenzyme Q10, including
storing a composition containing solid reduced coenzyme Q10 and water.
(2) The method according to (1), wherein the solid reduced coenzyme Q10 is one or more selected from the group consisting of a reduced coenzyme Q10 Form I crystal, a reduced coenzyme Q10 Form II crystal, a co-crystal composed of reduced coenzyme Q10 and one or more other compounds, and an amorphous solid of reduced coenzyme Q10.
(3) The method according to (1) or (2), wherein the composition is a liquid composition containing the solid reduced coenzyme Q10 in water or a solid composition containing the solid reduced coenzyme Q10 wetted with water.
(4) A method for controlling oxidation of reduced coenzyme Q10, including
storing a composition containing solid reduced coenzyme Q10 and water.
(5) The method according to (4), wherein the solid reduced coenzyme Q10 is one or more selected from the group consisting of a reduced coenzyme Q10 Form I crystal, a reduced coenzyme Q10 Form II crystal, a co-crystal composed of reduced coenzyme Q10 and one or more other compounds, and an amorphous solid of reduced coenzyme Q10.
(6) The method according to (4) or (5), wherein the composition is a liquid composition containing the solid reduced coenzyme Q10 in water or a solid composition containing the solid reduced coenzyme Q10 wetted with water.
(7) A liquid composition including solid reduced coenzyme Q10 in water.
(8) The liquid composition according to (7), including 10 parts by mass or more of water based on 1 part by mass of the solid reduced coenzyme Q10.
(9) The liquid composition according to (7) or (8), further including a surfactant.

The present description encompasses the specification and/or drawings in JP Patent Application Nos. 2021-210678 and 2022-152299 which serve as the basis of the priority of the present application.

### Advantageous Effects of Invention

According to the method for storing reduced coenzyme Q10 (QH) disclosed herein, oxidation of QH can be controlled and QH can be stably stored.

According to the method for controlling oxidation of QH disclosed herein, oxidation of QH can be effectively controlled.

According to the liquid composition including QH disclosed herein, oxidation of QH can be controlled and QH can be stably stored. Furthermore, solid QH can be easily separated from the liquid composition.

### DESCRIPTION OF EMBODIMENTS

Hereinafter, the present invention will be described in detail.

### <Reduced Coenzyme Q10>

“Reduced coenzyme Q10” in one or more embodiments of the present invention may partially include oxidized coenzyme Q10, provided that reduced coenzyme Q10 is included as a main component. The “main component” herein means that it is included in a percentage of, for example, 50% by weight or more, generally 60% by weight or more, preferably 70% by weight or more, more preferably 80% by weight or more, further preferably 90% by weight or more, particularly preferably 95% by weight or more, and further particularly 98% by weight or more. Herein, the above-described percentages are the percentages of reduced coenzyme Q10 to the total weight of coenzyme Q10

Besides, as mentioned above, reduced coenzyme Q10 has two forms of crystal polymorphisms, namely, Form I and Form II. Specifically, the crystal form of reduced coenzyme Q10 having a melting point around 48° C. and showing characteristic peaks at diffraction angles (2θ±0.2°) of 3.1°, 18.7°, 19.0°, 20.2°, and 23.0° in powder X-ray (Cu-Kα) diffraction is a Form I crystal, whereas the crystal form of reduced coenzyme Q10 having a melting point around 52° C. and showing characteristic peaks at diffraction angles (2θ±0.2°) of 11.5°, 18.2°, 19.3°, 22.3°, 23.0°, and 33.3° in powder X-ray (Cu-Kα) diffraction is a Form II crystal.

The “solid reduced coenzyme Q10” in one or more embodiments of the present invention can be preferably one or more selected from the group consisting of a reduced coenzyme Q10 Form I crystal, a reduced coenzyme Q10 Form II crystal, a co-crystal composed of reduced coenzyme Q10 and one or more other compounds, and an amorphous solid of reduced coenzyme Q10, particularly preferably one or more selected from the group consisting of a reduced coenzyme Q10 Form I crystal and a reduced coenzyme Q10 Form II crystal. The solid reduced coenzyme Q10 may be a solid further containing a component other than reduced coenzyme Q10, but is preferably a solid composed of reduced coenzyme Q10.

In the co-crystal composed of QH and one or more other compounds, such one or more other compounds included are not particularly limited as long as such each compound can form the co-crystal with QH, and examples thereof include organic carboxylic acids such as benzoic acid and derivatives thereof, organic alcohols such as resorcinol, benzyl alcohol and phenol, and derivatives thereof, urea, and nicotinamide. Such one or more other compounds may be one compound or two or more compounds, and are preferably one to three compounds.

The solid reduced coenzyme Q10 in one or more embodiments of the present invention is preferably a particulate solid. The size of the solid reduced coenzyme Q10 is not particularly limited, and the average particle size of the solid reduced coenzyme Q10 can be, for example, 1 μm or more, generally 2 μm or more, preferably 5 μm or more, more preferably 10 μm or more, and further preferably 30 μm or more, and can be generally 1 mm or less, preferably 0.5 mm or less, and more preferably 0.2 mm or less. The average particle size can be measured with a laser diffraction/scattering type particle size distribution measurement apparatus.

The solid reduced coenzyme Q10 (QH) used in one or more embodiments of the present invention is not required to be pre-formulated (i.e., need not be formulated in advance). In addition, solid QH is preferable because solid QH can be easily separated and recovered from a composition containing solid QH and water by a method such as filtration, centrifugation, or drying and thus QH can be utilized in a wide range of applications after storage. More preferably, solid QH is not pre-formulated QH (e.g., a clathrate of QH with cyclodextrin, QH dispersed in a matrix containing a water-soluble excipient in a particulate composition, QH coated with a coating material in a solid formulation, or a capsule of QH).

The water-soluble excipient can be, for example, one or more selected from the group consisting of a water-soluble polymer, a surfactant, sugar, and a yeast cell wall.

The coating material can be, for example, an oil-soluble coating material or a water-soluble coating material. The oil-soluble coating material can be, for example, a sugar ester of a higher fatty acid, shellac, a cellulose derivative, fatty acids and ester derivatives thereof, fats and oils, or zein. The water-soluble coating material can be, for example, gelatin, sugar, gum arabic, a sugar ester of a higher fatty acid, tragacanth, pectin, pullulan, alginic acid, dried albumen, milk, curdlan, a cellulose derivative, casein, a casein compound, starch, or a yeast cell wall.

The capsule is, for example, QH capsulated by a soft capsule, a hard capsule, a microcapsule, or the like. Examples of the material of the capsule may include gelatin derived from beef bone, bovine hide, pig hide, fishskin, or the like, a seaweed-derived product usable as a food additive, such as carrageenan or alginic acid; a plant seed-derived product such as locust bean gum or guar gum; an agent for production, containing cellulose; and starch such as wheat starch, potato starch, sweet potato starch, corn starch, or dextrin.

### <Method for Storing QH>

A first embodiment of the present invention relates to a method for storing QH, including storing a composition containing solid QH and water.

According to the method according to the present embodiment, oxidation of QH can be controlled and QH can be stably stored. In general, compounds are considered unstable in the presence of water, and it is considered important to store them in a dry state. The method according to the present embodiment utilizes the property that QH is stabilized in the state of being in contact with water, which is unique to QH and contrary to the above common belief, to allow QH to be stably stored. Preparation of a composition containing QH and water in contact with each other is easily carried out. Therefore, the method according to the present embodiment can serve as a measure for efficiently stabilizing QH at a low cost.

The solid QH is preferably one or more selected from the group consisting of a QH Form I crystal, a QH Form II crystal, a co-crystal composed of reduced coenzyme Q10 and one or more other compounds, and an amorphous solid of QH.

The QH Form II crystal, and the co-crystal composed of reduced coenzyme Q10 and one or more other compounds are themselves costly to produce, and therefore a QH residual rate after storage (refer to the definition to in Examples) of 80% or more is desired to provide QH in the above form at an appropriate price. The method according to the present embodiment is preferable in that the QH residual rate after storage can be 80% or more when QH is in the form of a QH Form II crystal or a co-crystal composed of reduced coenzyme Q10 and one or more other compounds.

QH in the form of a QH Form I crystal can be produced at a low cost, but is easily oxidized. Therefore, it is desired that the QH residual rate after storage (refer to the definition in Examples) is 40% or more in order to provide QH in the above form at an appropriate price. The method according to the present embodiment is preferable in that the QH residual rate after storage can be 40% or more when QH is in the form of a QH Form I crystal.

The composition containing solid QH and water in the method according to the present embodiment is not particularly limited as long as it contains solid QH and water, and examples of the composition containing solid QH and water may include a liquid composition containing solid QH in water and a solid composition containing solid QH wetted with water.

In the present embodiment, water may further contain any other component such as a surfactant or a salt.

The liquid composition containing solid QH in water is, for example, a liquid composition in which solid QH is suspended in water. The content of water in the liquid composition is not particularly limited, and is preferably 10 parts by mass or more and more preferably 30 parts by mass or more based on 1 part by mass of solid QH. The upper limit of the amount of water is not particularly limited, and can be, for example, 10,000 parts by mass or less, 1,000 parts by mass or less, or 100 parts by mass or less based on 1 part by mass of solid QH. In the liquid composition, water preferably further contains a surfactant. The liquid composition containing a surfactant can allow dispersion of solid QH to be stabilized. Examples of the surfactant may include polyglycerin fatty acid ester, polyalkylene glycol, alcohol, and alginate. The liquid composition can further contain other component, in addition to water and QH.

The content of water in the solid composition containing solid QH wetted with water is not particularly limited, and is preferably 0.05 parts by mass or more and more preferably 0.10 parts by mass or more based on 1 part by mass of solid QH. The upper limit of the amount of water is not particularly limited, and can be, for example, less than 10 parts by mass, 5 parts by mass or less, 3 parts by mass or less, or 2 parts by mass or less based on 1 part by mass of solid QH. The solid composition can further contain other component, in addition to water and QH.

The temperature at which the composition is stored in the method according to the present embodiment is, for example, a temperature of −25° C. or more and 50° C. or less, preferably a temperature of −20° C. or more, −10° C. or more, 0° C. or more, 4° C. or more, 10° C. or more, 15° C. or more, 20° C. or more, or 25° C. or more, and preferably a temperature of 45° C. or less, or 40° C. or less.

The period for which the composition is stored in the method according to the present embodiment is not particularly limited as long as it is a period until use of a product after production, and can be appropriately adjusted depending on storage conditions such as temperature. The period is preferably 3 days or more, 1 week or more, or 2 weeks or more and can be, for example, 5 years or less, generally 3 years or less, preferably 2 years or less, more preferably 1 year or less, further preferably 6 months or less, further preferably 8 weeks or less, and most preferably 6 weeks or less, 5 weeks or less, or 4 weeks or less.

When the composition is stored in the method according to the present embodiment, the composition may be a packaged body accommodated in a container and tightly sealed to prevent volatilization of water. The packaged body may include a gas phase in the container, and the gas phase may be air. The packaged body including air as the gas phase is preferable because it can be produced at a low cost compared to a packaged body including an inert gas such as nitrogen as the gas phase.

The method according to the present embodiment preferably further includes separating solid QH from the composition after storage. Solid QH separated can be used in various intended applications. Examples of the method for separating solid QH from the composition after storage include methods such as filtration, centrifugation, and drying.

### <Method for Controlling Oxidation of QH>

A second embodiment of the present invention relates to a method for controlling oxidation of QH, including storing a composition containing solid QH and water.

According to the method according to the present embodiment, oxidation of QH can be efficiently controlled at a low cost.

Respective characteristics and preferred aspects of solid QH, water, the composition, and the step of storing the composition in the method according to the present embodiment are as described with respect to the method according to the first embodiment.

The method according to the present embodiment preferably further includes separating solid QH from the composition after storage. Solid QH separated can be used in various intended applications. Examples of the method for separating solid QH from the composition after storage include methods such as filtration, centrifugation, and drying.

### <Liquid Composition>

A third embodiment of the present invention relates to a liquid composition containing solid reduced coenzyme Q10 in water.

Oxidation of QH in the form of the liquid composition according to the present embodiment is effectively controlled. Furthermore, solid QH can be easily separated from the liquid composition.

Respective characteristics and preferred aspects of solid QH, water, and the liquid composition in the liquid composition according to the present embodiment are as described with respect to the method according to the first embodiment.

### EXAMPLES

### 1. Raw Materials

The present invention will be further specifically described with reference to the following Examples, but the present invention is not limited to these Examples. In Examples, reduced coenzyme Q10 (trade name: Kaneka QH) manufactured by KANEKA CORPORATION was used as a reduced coenzyme Q10 Form I crystal (QH Form I crystal).

### 2. Method for Evaluating Oxidation Stability

The weight ratio of reduced coenzyme Q10 to total coenzyme Q10 (namely, reduced coenzyme Q10/(oxidized coenzyme Q10+reduced coenzyme Q10)) is defined as “QH ratio”. The QH ratio was determined by the following HPLC analysis. For the evaluation of oxidation stability, a rate of the change in the QH ratio at the end of the evaluation from that at the beginning of the evaluation was defined as “QH residual rate”, and the QH residual rate determined by the following expression was used as a measure of oxidation stability.

QH residual rate (%)=100×QH ratio at the end of the evaluation/QH ratio at the beginning of the evaluation

### (HPLC Analysis Conditions)

Column: SYMMETRY C18 (manufactured by Waters); 250 mm (length), 4.6 mm (inner diameter)
Mobile phase: C₂H₅OH: CH₃OH=4:3 (v:v)
Detection wavelength: 210 nm
Flow rate: 1 mL/min

### 3. Method for Preparing Reduced Coenzyme Q10 Form II Crystal (QH Form II Crystal)

To 611 g of ethanol, 89 g of the QH Form I crystal was added, and heated to 50° C. to completely dissolve the QH Form I crystal. This solution was cooled, and 1.8 g of a reduced coenzyme Q10 Form II crystal prepared according to the description of Patent Literature 2 was added as a seed crystal when the temperature of the solution reached 36° C. The solution was cooled to 33.5° C. over 7 hours, thereafter cooled to 25° C. at a rate of 1° C./hour, and further cooled to 1° C. at a rate of 10° C./hour to obtain a white slurry. The slurry obtained was filtered under reduced pressure to obtain a wet crystal, and the wet crystal was washed with cold ethanol and further dried under reduced pressure to obtain a QH Form II crystal.

4. Example of Storage of Reduced Coenzyme Q10 in Contact with Water

### Example 1

In a glass vial (volume 33 ml), 0.1 g of the QH Form I crystal was placed. Water or each aqueous solution listed in Table 1 was added to the glass vial in the amount shown in Table 1, and mixed with the QH Form I crystal. The glass vial was sealed, and stored under a temperature of 40° C. and a relative humidity of 75% for 2 weeks, after which the QH residual rate was determined.

TABLE 1

| | | Amount |
|---|---|---|
| | Water or aqueous solution | (g) |
| (1) | Water | 0.1 |
| (2) | 0.04% aqueous solution of hexaglycerin monolaurate | 0.01 |
| (3) | 0.04% aqueous solution of hexaglycerin monolaurate | 0.1 |
| (4) | 0.04% aqueous solution of hexaglycerin monolaurate | 3 |
| (5) | 1% aqueous solution of sodium alginate | 3 |
| (6) | 10% aqueous solution of glycerin | 0.1 |
| (7) | 10% aqueous solution of glycerin | 3 |
| (8) | 10% aqueous solution of polyethylene glycol 400 | 0.1 |
| (9) | 10% aqueous solution of polyethylene glycol 400 | 3 |

### Comparative Example 1

In a glass vial, 0.2 g of the QH Form I crystal was placed. The glass vial was opened, and stored under a temperature of 40° C. and a relative humidity of 11% for 2 weeks, after which the QH residual rate was determined.

The results obtained in Example 1 and Comparative Example 1 are summarized and shown in Table 2.

TABLE 2

| | | QH |
|---|---|---|
| | Storage | residual |
| | temperature | rate |
| | (° C.) | (%) |
| Example 1-(1) | 40 | 63.5 |
| Example 1-(2) | 40 | 67.5 |
| Example 1-(3) | 40 | 71.7 |
| Example 1-(4) | 40 | 84.6 |
| Example 1-(5) | 40 | 68.5 |
| Example 1-(6) | 40 | 64.0 |
| Example 1-(7) | 40 | 60.4 |
| Example 1-(8) | 40 | 70.5 |
| Example 1-(9) | 40 | 73.3 |
| Comparative | 40 | 34.9 |
| Example 1 | | |

### Example 2

In a glass vial (volume 33 ml), 0.1 g of the QH Form I crystal was placed. Each aqueous solution listed in Table 3 was added to the glass vial in the amount shown in Table 3, and mixed with the QH Form I crystal. The glass vial was sealed, and stored under a temperature of 25° C. and a relative humidity of 60% for 4 weeks, after which the QH residual rate was determined.

TABLE 3

| | | Amount |
|---|---|---|
| | Aqueous solution | (g) |
| (1) | 0.04% aqueous solution of hexaglycerin monolaurate | 3 |
| (2) | 0.04% aqueous solution of diglycerin monooleate | 3 |
| (3) | 20% aqueous solution of ethanol | 0.1 |
| (4) | 20% aqueous solution of ethanol | 3 |
| (5) | 10% aqueous solution of glycerin | 0.1 |
| (6) | 10% aqueous solution of glycerin | 3 |
| (7) | 10% aqueous solution of polyethylene glycol 400 | 0.1 |
| (8) | 10% aqueous solution of polyethylene glycol 400 | 3 |

### Comparative Example 2

In a glass vial (volume 33 ml), 0.1 g of the QH Form I crystal was placed. The glass vial was sealed, and stored under a temperature of 25° C. and a relative humidity of 60% for 4 weeks, after which the QH residual rate was determined.

The results obtained in Example 2 and Comparative Example 2 are summarized and shown in Table 4.

TABLE 4

| | | QH |
|---|---|---|
| | Storage | residual |
| | temperature | rate |
| | (° C.) | (%) |
| Example 2-(1) | 25 | 61.5 |
| Example 2-(2) | 25 | 60.8 |
| Example 2-(3) | 25 | 49.7 |
| Example 2-(4) | 25 | 67.0 |
| Example 2-(5) | 25 | 50.2 |
| Example 2-(6) | 25 | 54.5 |
| Example 2-(7) | 25 | 53.8 |
| Example 2-(8) | 25 | 61.8 |
| Comparative | 25 | 33.3 |
| Example 2 | | |

Tables 2 and 4 show that reduced coenzyme Q10 can be stably stored in contact with water and/or a water-containing substance. When the water and/or water-containing substance were/was in an amount of 0.1 parts by mass or more relative to reduced coenzyme Q10, reduced coenzyme Q10 was stably stored. It has also been found that the water-containing substance may contain a surfactant and that reduced coenzyme Q10 can be stabilized regardless of the type of the surfactant. Even in a case in which the water-containing substance was water-containing alcohol, reduced coenzyme Q10 was stably stored.

### Example 3

In a glass vial (volume 33 ml), 0.1 g of the QH Form II crystal was placed. 3 g of a 0.04% aqueous solution of hexaglycerin monolaurate was added to the glass vial, and mixed with the QH Form II crystal. The glass vial was sealed, and stored under a temperature of 40° C. and a relative humidity of 75% for 4 weeks, after which the QH residual rate was determined. As a result, the QH residual rate was 83.0%.

### Example 4

In a glass vial (volume 33 ml), 0.1 g of the QH Form II crystal was placed. 3 g of a 0.04% aqueous solution of hexaglycerin monolaurate was added to the glass vial, and mixed with the QH Form II crystal. The glass vial was sealed, and stored under a temperature of 25° C. and a relative humidity of 60% for 4 weeks, after which the QH residual rate was determined. As a result, the QH residual rate was 93.8%.

It has been revealed from Examples 3 and 4 that the QH Form II crystal is also stabilized in contact with water.

All publications, patents, and patent applications cited in the present description are incorporated herein by reference in their entirety.

The upper and/or lower limits of the numerical ranges described herein can be combined arbitrarily to define a preferred range. For example, a preferred range can be defined by arbitrarily combining the upper and lower limits of the numerical ranges, a preferred range can be defined by arbitrarily combining the upper limits of the numerical ranges, and a preferred range can be defined by arbitrarily combining the lower limits of the numerical ranges. The numerical range represented with the term “to” in the present application includes respective numerical values described before and after the term “to” as the upper and lower limits thereof.

It should be understood that throughout the present description, expressions in the singular forms include the concept of their plural forms, unless otherwise specifically stated. Thus, articles in the singular forms (e.g., “a,” “an,” and “the” in the English language) should be understood to include the concept of their plural forms, unless otherwise specifically stated.

Although the embodiment of the present invention has been described in detail above, the specific configuration is not limited to this embodiment, and even when there are design changes within the scope of the present disclosure, they are included in the present disclosure.

1. A method for storing reduced coenzyme Q10, comprising storing a composition comprising solid reduced coenzyme Q10 and water.

2. The method according to claim 1, wherein the solid reduced coenzyme Q10 is one or more selected from the group consisting of a reduced coenzyme Q10 Form I crystal, a reduced coenzyme Q10 Form II crystal, a co-crystal comprising reduced coenzyme Q10 and one or more other compounds, and an amorphous solid of reduced coenzyme Q10.

3. The method according to claim 1, wherein the composition is a liquid composition comprising the solid reduced coenzyme Q10 in water or a solid composition comprising the solid reduced coenzyme Q10 wetted with water.

4. A method for controlling oxidation of reduced coenzyme Q10, comprising
storing a composition comprising solid reduced coenzyme Q10 and water.

5. The method according to claim 4, wherein the solid reduced coenzyme Q10 is one or more selected from the group consisting of a reduced coenzyme Q10 Form I crystal, a reduced coenzyme Q10 Form II crystal, a co-crystal comprising reduced coenzyme Q10 and one or more other compounds, and an amorphous solid of reduced coenzyme Q10.

6. The method according to claim 4, wherein the composition is a liquid composition comprising the solid reduced coenzyme Q10 in water or a solid composition comprising the solid reduced coenzyme Q10 wetted with water.

7. A liquid composition comprising solid reduced coenzyme Q10 and water.

8. The liquid composition according to claim 7, comprising 10 parts by mass or more of water based on 1 part by mass of the solid reduced coenzyme Q10.

9. The liquid composition according to claim 7, further comprising a surfactant.

10. The method according to claim 1, wherein the solid reduced coenzyme Q10, prior to storing, is not pre-formulated with a clathrate forming material or a water-soluble excipient, is not coated with a coating material in a solid formulation, and is not present in a capsule of reduced coenzyme Q10.
